(19) United States
(12) Patent Application Publication
(10) Pub. No.: US 2008/0103124 A1
(43) Pub. Date: May 1, 2008

(54) Cefdinir-containing pharmaceutical composition

(75) Inventors: Tatsunobu Yoshioka, Tokyo (JP); Yoshiyuki Murakami, Tokyo (JP); Noboru Yamashita, Tokyo (JP); Shigemitsu Tomei, Tokyo (JP); Katsumi Saito, Tokyo (JP); Akira Takagi, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: ASTELLAS PHARMA INC.

(21) Appl. No.: 11/976,351

(22) Filed: Oct. 24, 2007

Related U.S. Application Data
(60) Provisional application No. 60/854,082, filed on Oct. 25, 2006.

(51) Int. Cl. A61K 31/00; A61P 43/00

(52) U.S. Cl. 514/210.05

(57) ABSTRACT

A pharmaceutical composition with an enhanced bioavailability, particularly improved an oral absorption, comprising cefdinir or a pharmaceutically acceptable salt thereof and aminoalkyl methacrylate copolymer E is disclosed.

### CROSS REFERENCE TO RELATED APPLICATION

This application claims the benefit of U.S. Provisional Application No. 60/854,082, filed Oct. 25, 2006, the content of which is incorporated herein by reference.

### BACKGROUND OF THE INVENTION

1. Field of the Invention

The present invention relates to a pharmaceutical composition with an enhanced bioavailability (particularly, an improved oral absorption) of cefdinir or a pharmaceutically acceptable salt thereof. More particularly, the present invention relates to a pharmaceutical composition with an enhanced bioavailability (particularly, an improved oral absorption) comprising cefdinir or a pharmaceutically acceptable salt thereof, aminoalkyl methacrylate copolymer E, and if desired, an acidic substance, and relates to a pharmaceutical composition with an enhanced bioavailability (particularly, an improved oral absorption) comprising cefdinir or a pharmaceutically acceptable salt thereof and pulverized aminoalkyl methacrylate copolymer E.

2. Description of the Related Art

Cefdinir [chemical name: (6R, 7R)-7-[(Z9-2-(2-Aminothiazol-4-yl)-2-(hydroxyimino)acetylamino]-8-oxo-3-vinyl-5-thia-1-azabicyclo[4.2.0]oct-2-ene-2-carboxylic acid] is known as a potent β-lactam antibiotic (patent reference 1).

In general, the oral administration of antibiotics sometimes causes side effects on the digestive system such as diarrhea. To resolve this problem, a reduction of the side effects on the digestive system has been attempted, while maintaining the bioavailability of conventional pharmaceutical preparations. As such an attempt, for example, a sustained release of a drug was attempted, to lower the maximum blood drug concentration and the variability index in plasma concentration, while maintaining the bioavailability of a conventional pharmaceutical preparation (patent reference 2 and patent reference 3). More particularly, in pharmaceutical preparations with an improved absorption containing macrolide antibiotics, erythromycin (patent reference 2) or azithromycin (patent reference 3), a sustained release was achieved by adding hydroxypropylmethylcellulose to the preparations, to reduce the side effects thereof.

As a cefdinir formulation with an improved oral absorption, a pharmaceutical composition containing cefdinir and a bile acid or a sucrose fatty acid ester is known (patent reference 4 and patent reference 5).

As a pharmaceutical composition containing aminoalkyl methacrylate copolymer E, a pharmaceutical composition with an improved oral absorption containing a drug, aminoalkyl methacrylate copolymer E, and an acidic substance (patent reference 6), and an antibacterial composition in which a macrolide antibiotic, 4″-O-(paramethoxyphenylacetyl)tyrosine, is amorphously dispersed in aminoalkyl methacrylate copolymer E, obtained by dissolving aminoalkyl methacrylate copolymer E in the macrolide antibiotic solution and spray-drying the solution with a spray-dryer (patent reference 7) are known. In the antibacterial composition disclosed in patent reference 7, 4″-O-(paramethoxyphenylacetyl)tyrosine is formed as a solid dispersion to enhance the solubility thereof, and thus, the absorption thereof is improved.

However, an improvement of the cefdinir absorption by aminoalkyl methacrylate copolymer E has not been reported.
[patent reference 1] Japanese Unexamined Patent Publication (Kokai) No. 59-89689
[patent reference 2] WO 98/46239
[patent reference 3] WO 95/30422
[patent reference 4] Japanese Unexamined Patent Publication (Kokai) No. 62-265226
[patent reference 5] Japanese Unexamined Patent Publication (Kokai) No. 1-128926
[patent reference 6] WO 2002/005786
[patent reference 7] EP 413229/Japanese Unexamined Patent Publication (Kokai) No. 3-74396

### SUMMARY OF THE INVENTION

The absolute bioavailability of cefdinir in human beings is 21% following the administration of a 300 mg capsule, and 25% following the administration of an oral suspension of 250 mg/5 mL, in accordance with a package insert attached to cefdinir preparations [OMNICEF (trade name); Abbott Laboratories] which is commercially available in the United States, and the bioavailability of cefdinir is not high in either of these forms.

An object of the present invention is to enhance the bioavailability of cefdinir preparations, that is, to provide a preparation in which, even if a content of cefdinir in the preparation is lowered in comparison with that in conventional preparations, the pharmacological activity thereof (and the concentration in blood) similar to those of conventional preparations will be maintained to reduce side effects on the digestive system.

To solve this object, the present inventors attempted the following various approaches, but could not obtain the desired results. The present inventors attempted one more approach from a different viewpoint, to find that only aminoalkyl methacrylate copolymer E, among the following various agents capable for absorption enhancement, had a desired activity of improving oral absorption, and completed the present invention.

First, an effect of the particle size of cefdinir on bioavailability was examined in human beings. Differences in the particle size did not affect the bioavailability, and the bioavailability was similar to that following administration of solution of cefdiner.

Next, a preparation containing hydroxypropylmethylcellulose [HPMC; TC5E (trade name); Shin-Etsu Chemical Co., Ltd.)] and polyoxyethylene-hydrogenated castor oil (HCO-60), which were known as agents for improving the solubility of a base, was attempted, but the desired results were not obtained. In this connection, the amounts of HPMC and HCO-60 were 3 parts and 0.5 parts, respectively, with respect to 1 part of cefdinir.

From a viewpoint of an inhibition of inactivation in the intestines, trehalose or/and tannic acid was added to attempt a formation of a complex with an iron ion, which was known to bind with cefdinir to inhibit the absorption of cefdinir, but the desired results were not obtained.

A promotion of intestinal absorption by utilizing PEPT1, which had been reported as a transporter of cefdinir, was examined. A change in pH did not affect the absorption, and an inhibitory effect was not observed even when a substrate of PEPT1 (glycylsarcosine) was simultaneously added.

To carry out a paracellular opening by trapping calcium ions from tight-junctions in the mucous membrane of the digestive tract, an effect of EDTA was examined, but it was difficult to improve the absorption.

The present inventors assumed that the decreased bioavailability was caused by the efflux transport of absorbed cefdinir into the digestive tract by an anion transporter. From a viewpoint of an inhibition of the efflux into the digestive tract, probenecid or benzoic acid was added to attempt a competitive inhibition, but any significant results were not observed.

In view of an inhibition of elimination, an elimination inhibition by probenecid was examined, but the inhibition was not observed after a simultaneous administration.

As described above, the desired effects were not obtained by these approaches. The present inventors used various compounds known as agents capable of promoting absorption, that is, glycine, glucose, sodium citrate, polyethylene glycol 400, TPGS [Tocopheryl Poly(ethylene glycol 1000) Succinate], polysorbate 80 (Tween 80), a combination of sorbitan monolaurate (Span 20) and sodium lauryl sulfate (SLS), saturated polyglycolysed glyceride [polyglycolysed C8-C8 glyceride, Gelucire (trade name); Gattefossé], polyglycolysed glyceride [PEG-8 glyceryl caprylate/caprate, Labrasol (trade name); Gattefossé], a combination of caprylic/capric triglyceride [Panacet 810 (trade name); NOF Corporation] and SLS, sucrose stearate ester [DK F-160 (trade name); Dai-ichi Kogyo Seiyaku Co., Ltd.)], a combination of capric monoglyceride and SLS, a combination of stearic acid and SLS, and aminoalkyl methacrylate copolymer E, to examine an effect on absorption improvement using a rat intestinal loop experiment. As a result, it was found that only the aminoalkyl methacrylate copolymer E had a desired activity of absorption enhancement.

The present invention relates to
[1] a pharmaceutical composition comprising cefdinir or a pharmaceutically acceptable salt thereof and aminoalkyl methacrylate copolymer E;
[2] the pharmaceutical composition of [1], further comprising an acidic substance;
[3] the pharmaceutical composition of [1] or [2], wherein the aminoalkyl methacrylate copolymer E is pulverized;
[4] the pharmaceutical composition of [2] or [3], comprising cefdinir or a pharmaceutically acceptable salt thereof, the aminoalkyl methacrylate copolymer E, and an acidic substance, wherein the three components are brought together and at least the aminoalkyl methacrylate copolymer E and the acidic substance are uniformly mixed;
[5] the pharmaceutical composition of [4], wherein cefdinir or a pharmaceutically acceptable salt thereof, the aminoalkyl methacrylate copolymer E, and an acidic substance are uniformly mixed;
[6] the pharmaceutical composition of [1] to [5], wherein an amount of the aminoalkyl methacrylate copolymer E is two or more parts by weight with respect to one part by weight of cefdinir or a pharmaceutically acceptable salt thereof;
[7] the pharmaceutical composition of [2] to [6], wherein the acidic substance has a feature such that when 1 g of the acidic substance is dissolved in 50 mL of water, a pH of the solution is 6 or lower;
[8] the pharmaceutical composition of [2] to [7], wherein the acidic substance is contained in an amount which neutralizes 10% or more of basic groups contained in the aminoalkyl methacrylate copolymer E;
[9] the pharmaceutical composition of [2] to [8], comprising 2 to 500 parts by weight of the aminoalkyl methacrylate copolymer E with respect to one part by weight of cefdinir or a pharmaceutically acceptable salt thereof in an amount effective for treating or preventing disease, and the acidic substance in an amount which neutralizes 10% or more of basic groups contained in the aminoalkyl methacrylate copolymer E;
[10] the pharmaceutical composition of [2] to [9], comprising 2 to 500 parts by weight of the aminoalkyl methacrylate copolymer E with respect to one part by weight of cefdinir or a pharmaceutically acceptable salt thereof in an amount effective for treating or preventing disease, and 0.005 to 50 parts by weight of the acidic substance with respect to one part by weight of the aminoalkyl methacrylate copolymer E;
[11] the pharmaceutical composition of [2] to [10], wherein the aminoalkyl methacrylate copolymer E and the acidic substance are obtained by spray-drying or freeze-drying a solution and/or suspension thereof in a pharmaceutically acceptable solvent;
[12] the pharmaceutical composition of [2] to [11], wherein the aminoalkyl methacrylate copolymer E and the acidic substance are contained in a form of a solution and/or suspension thereof in a pharmaceutically acceptable solvent;
[13] the pharmaceutical composition of [1] to [12], wherein the form of the pharmaceutical composition is one, or two or more preparations selected from the group consisting of granules, tablets, capsules, a suspension, and a liquid formulation;
[14] a method of enhancing the bioavailability of cefdinir or a pharmaceutically acceptable salt thereof comprising: mixing cefdinir or a pharmaceutically acceptable salt thereof with the aminoalkyl methacrylate copolymer E;
[15] the method of [14], wherein an acidic substance is further added in the mixing step;
[16] the method of [14] or [15], wherein the aminoalkyl methacrylate copolymer E is pulvelized;
[17] the method of [14] to [16], wherein the enhancement of the bioavailability is an improvement of oral absorption;
[18] a use of aminoalkyl methacrylate copolymer E as an agent for enhancing the bioavailability of cefdinir or a pharmaceutically acceptable salt thereof;
[19] a use of aminoalkyl methacrylate copolymer E and an acidic substance as an agent for enhancing the bioavailability of cefdinir or a pharmaceutically acceptable salt thereof;
[20] the use of [18] or [19], wherein the aminoalkyl methacrylate copolymer E is pulvelized; and
[21] the use of [18] to [20], wherein the agent for enhancing the bioavailability is an agent for improving oral absorption.

According to the present invention, the bioavailability of cefdinir preparations can be enhanced. Therefore, even when a content of cefdinir in a preparation is lowered in comparison with that in conventional preparations, the pharmacological activity (and the blood concentration) can be maintained at a level similar to those of conventional preparations, and side effects on the digestive system can be reduced.

### BRIEF DESCRIPTION OF DRAWINGS

FIG. 1 is a graph showing time courses in mean plasma concentrations (mean±SD; n=3) after an administration of cefdinir solutions (10 mg/kg) with E-SD (spray-dried aminoalkyl methacrylate copolymer E, see Example 1 for datails) to a rat intestinal loop. The horizontal axis indicates a time (hr), and the vertical axis indicates a plasma concentration of cefdinir (ng/mL).

### DESCRIPTION OF THE PREFERRED EMBODIMENTS

The cefdinir which may be used in the present invention is a compound [chemical name: (6R, 7R)-7-[(Z9-2-(2-Aminothiazol-4-y1)-2-(hydroxyimino)acetylamino]-8-oxo-3-vinyl-5-thia-1-azabicyclo[4.2.0]oct-2-ene-2-carboxylic acid] of the formula:

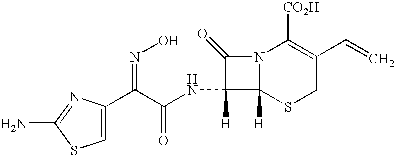

and is known as a potent β-lactam antibiotic [for example, Japanese Unexamined Patent Publication (Kokai) No. 59-89689 (particularly, Examples 14 and 16), and Japanese Unexamined Patent Publication (Kokai) No. 1-250384].

Cefdinir is commercially available, for example, as capsules (300 mg/dose, twice a day, or 600 mg/dose, once a day in the United States; and 100 mg/dose, three times a day in Japan), an oral suspension (7 mg/kg/dose, twice a day, or 14 mg/kg, once a day in the United States) and granules (3 to 6 mg/kg/dose, three times a day in Japan) for pediatric subjects.

In the present invention, the content of cefdinir or a pharmaceutically acceptable salt thereof contained in the pharmaceutical compound is not particularly limited, so long as it is an amount effective for a treatment. Capsules may contain the active ingredient at a daily dose of 200 to 600 mg, preferably 300 to 550 mg, more preferably 400 to 500 mg in the United States, and at a daily dose of 100 to 300 mg, preferably 150 to 275 mg, more preferably 200 to 250 mg in Japan. An oral suspension may contain the active ingredient at a daily dose of 4.7 to 14 mg/kg, preferably 7 to 12.8 mg/kg, more preferably 9.3 to 11.7 mg/kg in the United States. Pediatric granules may contain the active ingredient at a daily dose of 3 to 18 mg/kg, preferably 4.5 to 16.5 mg/kg, more preferably 6 to 15 mg/kg.

Cefdinir is active against, for example, Staphylococcus, Streptococcus, Streptococcus pneumoniae, Neisseria gonorrhoeae, Moraxella (Branhamella) catarrhalis, Escherichia coli, Citrobacter, Klebsiella, Enterobacter, Serratia, Proteus, Morganella morganii, Providencia, Haemophilus influenzae, Peptostreptococcus, Bacteroides, Prevotella (excluding Prevotella bivia), or Propionibacterium acnes. Cefdinir is effective in the treatment of, for example, superficial skin infection, deep skin infection, lymphangitis and lymphadenitis, chronic pyoderma/secondary infection, mastitis, and perirectal abscess caused by trauma, burns, operative wounds or the like/secondary infection caused by pharyngolaryngitis, tonsillitis (including peritonsillitis and peritonsillar abscess), acute bronchitis, pneumonia, or chronic respiratory lesions/cystitis, pyelonephritis/urethritis, cervicitis/cholecystitis, cholangitis/bartholinitis, intrauterine infection, adnexitis/dacryocystitis, hordeolum, tarsadenitis/otitis externa, otitis media, sinusitis/periodontal tissue inflammation, pericoronitis, or gnathitis.

Cefdinir or a pharmaceutically acceptable salt thereof may be used in any state, such as an amorphous state or a crystalline state. From the viewpoint of stability, the crystalline state [Japanese Unexamined Patent Publication (Kokai) No. 1-250384] is preferred to the amorphous state. Further, the crystalline state is preferable as the cefdinir or a pharmaceutically acceptable salt thereof contained in the pharmaceutical preparation.

The aminoalkyl methacrylate copolymer E used in the present invention is a copolymer of methyl methacrylate, butyl methacrylate, and dimethylaminoethyl methacrylate. The aminoalkyl methacrylate copolymer E was developed by Röhm GmbH, and is commercially available, for example, in the trade name of Eudragit™ E100 (Röhm GmbH). Pulvelized aminoalkyl methacrylate copolymer E is commercially available in the trade name of Eudragit™ EPO (Röhm GmbH). Eudragit™ E100 has the properties of rapidly dissolving in gastric juices, dissolving in a buffer having a pH of 5.0 or lower, and swelling film in a buffer having a pH of 5.0 or higher. Eudragit™ EPO is fine powder obtained by pulvelizing aminoalkyl methacrylate copolymer E, and exhibits an improved solubility (rate of dissolution) and dispersibility.

A state of aminoalkyl methacrylate copolymer E contained in the pharmaceutical composition of the present invention is not particularly limited, so long as the aminoalkyl methacrylate copolymer E is brought together with cefdinir or a pharmaceutically acceptable salt thereof, and these components are uniformly mixed, with an acid substance that may be added if desired. As such a state, there may be mentioned, for example, a solid such as a powder of the copolymer, or a liquid obtained by suspending and/or dissolving the copolymer in water. The powder may be prepared by conventional methods, such as pulverizing, spray-drying, lyophilization, wet granulation, or dry granulation. It is preferable that an acid substance, which will be mentioned below, is added as a solubilizing agent for the copolymer. The aminoalkyl methacrylate copolymer E may have free amino groups, and may be a soluble salt. When the aminoalkyl methacrylate copolymer E is a soluble salt, a preferred embodiment is a preparation obtained by spray-drying or lyophilizing a solution or suspension of the aminoalkyl methacrylate copolymer E and acid.

The aminoalkyl methacrylate copolymer E may be used together with a surfactant. The surfactant to be added is not particularly limited, so long as it is pharmaceutically acceptable and is capable of reducing the water repellency of the copolymer. As the surfactant, there may be mentioned, for example, nonionic surfactants [for example, polyoxyethylene surfactants (such as polysorbate 80 (Tween 80), polyoxyl stearate 40, lauromacrogol, or polyoxyethylene-hydrogenated castor oil (HCO-60)), or sucrose fatty acid ester], or ionic surfactants [anionic surfactants (for example, sodium lauryl sulfate), cationic surfactants (for example, benzalkonium chloride), or amphoteric surfactants (for example, lecithin)]. These surfactants may be used alone, or as a combination of two or more surfactants. The amount of the surfactant to be added is not particularly limited, so long as it is an amount which can reduce the water repellency of the copolymer. The amount of the surfactant is usually approximately 0.01 to 10 parts by weight, preferably approximately 0.01 to 5 parts by weight, more preferably approximately 0.05 to 1 part by weight, with respect to 1 part by weight of the polymer.

A solvent in which the aminoalkyl methacrylate copolymer E is dissolved or suspended (if desired, together with a surfactant) is not particularly limited, so long as it is pharmaceutically acceptable. The solvent may be, for example, water, organic solvents (such as methanol, ethanol, isopropanol, or acetone), or a mixture of water and organic solvent(s). Further, the pharmaceutical composition of the present invention may contain various fillers that are used as pharmaceutical additives, and other additives. As the filler or additive, extenders such as lactose or starch may be added.

The amount of the aminoalkyl methacrylate copolymer E used in the present invention is not particularly limited, so long as it may be appropriately adjusted on the basis of the amount of cefdinir or a pharmaceutically acceptable salt thereof. The amount of the aminoalkyl methacrylate copolymer E is usually two or more parts by weight, preferably 2 to 500 parts by weight, more preferably 2 to 250 parts by weight, most preferably 2 to 50 parts by weight, with respect to one part by weight of cefdinir or a pharmaceutically acceptable salt thereof.

The acidic substance which may be used in the present invention is not particularly limited, so long as it is pharmaceutically acceptable and capable of dissolving the aminoalkyl methacrylate copolymer E by neutralizing some or all of the basic groups of the copolymer in the presence of water. As the acidic substance, an inorganic acid and/or an organic acid in which the pH of a solution prepared by dissolving or suspending 1 g of the substance in 50 mL of water is 6 or lower are preferred. As the acidic substance, there may be mentioned, for example, inorganic acids (such as hydrochloric acid, phosphoric acid, potassium dihydrogen phosphate, or sodium dihydrogen phosphate), organic acids (such as citric acid, lactic acid, tartaric acid, fumaric acid, phthalic acid, acetic acid, oxalic acid, malonic acid, adipic acid, phytic acid, succinic acid, glutaric acid, maleic acid, malic acid, mandelic acid, ascorbic acid, benzoic acid, methanesulfonic acid, capric acid, capronic acid, caprylic acid, lauric acid, arachidonic acid, erucic acid, linoleic acid, linolenic acid, palmitic acid, myristic acid, or stearic acid), aspartic acid, L-glutamic acid, L-cystein, arginine hydrochloride, lysine hydrochloride, or L-glutamic acid hydrochloride. These acidic substances may be used alone or as a combination thereof.

The content of the acidic substance used in the present invention is not particularly limited, so long as it is an amount capable of dissolving the aminoalkyl methacrylate copolymer E by neutralizing some or all of the basic groups of the copolymer in the presence of water. The amount of the acidic substance to be added is an amount which neutralizes approximately 10% or more, preferably approximately 15% or more, more preferably approximately 30% or more, still more preferably approximately 40% or more, most preferably 50% or more, of the basic groups of the copolymer. It is preferable that this value is 50% or more, because a spray-dried product can be easily handled during production without aggregation, even when stored for a long period of time. The amount of the acidic substance may be appropriately adjusted in accordance with the solubility and/or the acidity of the acidic substance, and it is generally 0.005 to 50 parts by weight, preferably 0.01 to 30 parts by weight, more preferably 0.03 to 10 parts by weight, with respect to 1 part by weight of the aminoalkyl methacrylate copolymer E.

For example, when 312.5 g of 1 mol/L hydrochloric acid is added, as the acidic substance used in the present invention, to 500 g of aminoalkyl methacrylate copolymer E, and the mixture is spray dried, the calculation may be carried out by the following equation (I):

(1×312.5)/1000 [a]=X/[KOH(56)] [b] (I)
[a: Number of moles of HCl, b: Number of moles of KOH]
X=17.49 g, but it is the amount in 500 g, and thus, this value is divided by 500.
X/1 g aminoalkyl methacrylate copolymer E=35 mg KOH
Actually, the alkali value in 1 g of aminoalkyl methacrylate copolymer E is 163 to 198 mg KOH, and thus, the amount of the acid added is 15 to 20% of the amount that neutralizes all of the alkali.

The uniform mixing of the aminoalkyl methacrylate copolymer E with the acidic substance used in the present invention is not particularly limited, so long as it is a state in which both are brought together with cefdinir or a pharmaceutically acceptable salt thereof and uniformly mixed, and the aminoalkyl methacrylate copolymer E can be dissolved by the acidic substance in the presence of water. A state in which the three components are uniformly mixed is preferred. Such states may be obtained by conventional methods. There may be mentioned, for example, a method using aminoalkyl methacrylate copolymer E prepared by the previously described method(s); a method in which aminoalkyl methacrylate copolymer E and the acidic substance, optionally with cefdinir or a pharmaceutically acceptable salt thereof, are dissolved and/or suspended in a pharmaceutically acceptable solvent [for example, water, alcohols (such as methanol, ethanol, propanol, or butanol), or a mixture thereof], and the resulting liquid is treated with a conventional method, such as spray drying, to convert it to powder; a method in which aminoalkyl methacrylate copolymer E and the acidic substance are mixed or granulated by a conventional method to obtain a mixture; a method in which aminoalkyl methacrylate copolymer E and the acidic substance are dissolved and/or suspended in a pharmaceutically acceptable solvent to obtain a liquid; or a method in which cefdinir or a pharmaceutically acceptable salt thereof is further added in these methods. According to these methods, although the aminoalkyl methacrylate copolymer E is not usually dissolved in a neutral buffer, a spray-dried product obtained from a mixed solution of aminoalkyl methacrylate copolymer E and the acidic substance can be dissolved in a neutral buffer.

The term “brought together” as used herein means a state in which each component of the drug, aminoalkyl methacrylate copolymer E, and the acidic substance is present and close to one another in a solid or liquid state. The concept of “brought together” includes a state in which each component is in contact with one another. Further, the state of the drug is not particularly limited, and the drug may be used without any treatment or after a pretreatment. When the stability of the drug is lowered by the contact with the acidic substance or the like, the drug is usually used after a pretreatment (for example, a coating with a water-soluble substance such as sugars, a starch, or hydroxypropylmethyl cellulose). This state in which the treated drug and the other components are present and close to one another or in contact with one another is also included in the concept of “brought together”. The state of being present and close to one another means a state in which each component is present to such an extent that the purpose of the present invention, that is, an improvement of drug permeability in the mucous layer and/or the mucous membrane of the digestive tract and an improvement of oral absorption, is achieved.

The term “at least” as used herein means two components of aminoalkyl methacrylate copolymer E and the acidic substance, or three components of the drug, aminoalkyl methacrylate copolymer E, and the acidic substance.

The term “uniformly” as used herein means a state in which each component of the drug, aminoalkyl methacrylate copolymer E, and the acidic substance is uniformly dispersed and present as a whole, that is, a state in which each component is not unevenly distributed. For example, a state in which each component is unevenly distributed, such as a three-layer tablet in which the drug, aminoalkyl methacrylate copolymer E, and the acidic substance are independently layered, is not included in the term “uniformly”. The term “uniformly mixed” as used herein means a state of being mixed by conventional methods known in the field of pharmaceutical preparations, for example, a solid composition in which each component is produced by physical mixing, spray drying, lyophilization, or granulation (such as wet granulation or dry granulation), or a liquid composition in which each component is suspended and/or dissolved in a pharmaceutically acceptable solvent such as water.

The form of the pharmaceutical composition is not particularly limited, so long as it can be orally administered. This preparation may be, for example, powder, tablets, capsules, a liquid, a suspension, an emulsion, or capsules filled with a liquid, a suspension, an emulsion, or the like, preferably a suspension. Alternatively, the preparation may be powder, granulated granules, or lyophilized cake, which may be used to prepare a liquid, a suspension, an emulsion, or the like by adding it to water or the like and stirring. These preparations may be produced by conventional methods.

Hereinafter, the pharmaceutical composition of the present invention will be further illustrated, on the basis of embodiments simultaneously containing the aminoalkyl methacrylate copolymer E and the acidic substance. The following descriptions can be applied to embodiments not containing the acidic substance, by replacing the term “aminoalkyl methacrylate copolymer E and the acidic substance” with “aminoalkyl methacrylate copolymer E” in accordance with technical common knowledge of those skilled in the art, unless otherwise specified.

A preparation in which the aminoalkyl methacrylate copolymer E and the acidic substance used in the present invention are present and close to cefdinir or a pharmaceutically acceptable salt thereof is preferable. As such a preparation, there may be mentioned, for example, a solution and/or a suspension prepared by dissolving or suspending the aminoalkyl methacrylate copolymer E and the acidic substance in a pharmaceutically acceptable solvent; capsules in which this solution/suspension is filled in capsules such as gelatin capsules; a mixture obtained by mixing the aminoalkyl methacrylate copolymer E and the acidic substance by a conventional method and then mixing this mixture with cefdinir or a pharmaceutically acceptable salt thereof; granules obtained by mixing the aminoalkyl methacrylate copolymer E and the acidic substance, adding a pharmaceutically acceptable solvent such as water, optionally adding a binder such as hydroxypropyl methyl cellulose, and granulating this mixture; tablets obtained by mixing a pharmaceutical filler with the above-mentioned mixture or granules, and tableting this mixture; capsules in which the above-mentioned granules are filled in, for example, gelatin capsules; an enteric coated preparation obtained by coating the above-mentioned granules with an enteric substance [for example, a 1:1 copolymer of methyl methacrylate and methacrylic acid (trade name: Eudragit™ L; Röhm GmbH), a 2:1 copolymer of methyl methacrylate and methacrylic acid (trade name: Eudragit™ S, Röhm GmbH), a 1:1 copolymer of ethyl acrylate and methacrylic acid (trade name: Eudragit™ LD-55, Röhm GmbH), hydroxypropyl methyl cellulose phthalate, hydroxypropyl methyl cellulose acetate succinate, carboxymethyl ethyl cellulose, cellulose acetate phthalate, shellac, or zein]; or an enteric coated preparation obtained by coating tablets previously formed by tableting the above-mentioned granules, with the enteric substance. These preparations may be produced by conventional methods. If desired, pharmaceutical additives, such as fillers, disintegrators, binders, lubricants, fluidizers, dispersants, suspension agents, emulsifiers, preservatives, or stabilizers, may be added to the pharmaceutical composition of the present invention.

When the pharmaceutical composition of the present invention is a liquid or a suspension, the liquid or suspension is prepared by suspending at least cefdinir or a pharmaceutically acceptable salt thereof, aminoalkyl methacrylate copolymer E, and the acidic substance in a pharmaceutically acceptable solvent, and may further contain one or more pharmaceutically acceptable additives. As the pharmaceutical additives, sweeteners, coloring agents, flavors, pH adjusting agents, thickening agents, suspension agents, stabilizers, or the like may be added. Alternatively, the liquid or suspension may be prepared as powder, a granular preparation such as granulated granules, or lyophilized cake, which may be used to prepare a liquid, a suspension, or the like by adding thereto a pharmaceutically acceptable solvent such as water and stirring before use. These preparations may contain pharmaceutical additives such as fillers, disintegrators, binders, lubricants, fluidizers, dispersants, suspension agents, emulsifiers, preservatives, stabilizers, sweeteners, coloring agents, flavors, pH adjusting agents, thickening agents, and suspension agents, if desired, and may be produced by conventional methods.

When the pharmaceutical composition is a granular preparation, the process thereof is not particularly limited. The granular preparation may be produced by conventional methods, such as a fluidized bed granulation, an agitation granulation, a high speed agitation granulation, an tumbling fluidized bed glanulation, or a dry granulation. For example, the granular preparation may be produced by uniformly mixing cefdinir or a pharmaceutically acceptable salt thereof, the aminoalkyl methacrylate copolymer E, the acidic substance, and pharmaceutical additive(s), compressing this mixture using a slug tablet machine or a roller compacter to form tablets, and crushing the tablets into granules using a granulator. In the crushing step, a granulator commonly used for crushing pharmaceutical preparations, such as an atomizer (such as a sample mill or a hammer mill), a pin mill, a jet mill, or a ball mill, may be used. In the mixing step of the production of the granular preparation, cefdinir or a pharmaceutically acceptable salt thereof, the aminoalkyl methacrylate copolymer E, the acidic substance, and pharmaceutical additive(s) may be mixed at a time, or stepwisely, that is, by mixing a part of these components and then mixing it uniformly with the remaining component(s). After the granulation, the granulated products may be classified by screen(s).

Alternatively, the granular preparation may be produced by uniformly mixing cefdinir or a pharmaceutically acceptable salt thereof, the aminoalkyl methacrylate copolymer E, the acidic substance, and pharmaceutical additive(s), and spraying a solvent in a fluidized bed granulator. The solvent is not particularly limited, but water, lower alcohols (for example, C₁₋₄ alcohols such as ethanol or isopropanol), aliphatic ketones such as acetone, or a mixture thereof may be commonly used. From the viewpoint of safety, water and/or ethanol (for example, ethanol alone, or a mixing solvent of water and ethanol) are preferred. Cefdinir or a pharmaceutically acceptable salt thereof, the aminoalkyl methacrylate copolymer E, the acidic substance, and pharmaceutical additive(s) may be mixed at a time and granulated, or a part of these components may be mixed, and then the remaining component(s) dissolved or suspended in a solvent may be sprayed to obtain granules. After the granulation, the granulated products may be classified by screen(s).

The pharmaceutical composition may contain, with respect to 1 part by weight of cefdinir or a pharmaceutically acceptable salt thereof in an amount effective for treating or preventing disease, 2 to 500 parts by weight (preferably 2 to 250 parts by weight, more preferably 2 to 50 parts by weight) of the aminoalkyl methacrylate copolymer E, and the acidic substance in an amount which neutralizes 10% or more (preferably 15% or more, more preferably 30% or more, still more preferably 40% or more, most preferably 50% or more) of the basic groups contained in this copolymer. The mixture ratio may be selected, as an appropriate combination, from the groups consisting of preferable mixture ratios of each component. The most preferable mixture ratio is, with respect to 1 part by weight of cefdinir or a pharmaceutically acceptable salt thereof in an amount effective for treating or preventing disease, 2 to 50 parts by weight of the aminoalkyl methacrylate copolymer E, and the acidic substance in an amount which neutralizes 50% or more of the basic groups contained in this copolymer. The mixture ratio of the three components in the pharmaceutical composition is 2 to 500 parts by weight (preferably 2 to 250 parts by weight, more preferably 2 to 50 parts by weight) of the aminoalkyl methacrylate copolymer E with respect to 1 part by weight of cefdinir or a pharmaceutically acceptable salt thereof in an amount effective for treating or preventing disease, and 0.005 to 50 parts by weight (preferably 0.01 to 30 parts by weight, more preferably 0.03 to 10 parts by weight) of the acidic substance with respect to 1 part by weight of the copolymer. The mixture ratio may be selected, as an appropriate combination, from the groups consisting of preferable mixture ratios of each component. The most preferable mixture ratio is 2 to 50 parts by weight of the aminoalkyl methacrylate copolymer E with respect to 1 part by weight of cefdinir or a pharmaceutically acceptable salt thereof in an amount effective for treating or preventing disease, and 0.03 to 10 parts by weight of the acidic substance with respect to 1 part by weight of the copolymer.

The pharmaceutical composition of the present invention may be applied to various preparations, as described above. As such preparations, there may be mentioned, for example, sustained-release pharmaceutical preparations (for example, see International Publication Pamphlet WO 94/06414), timed-release or pulsed-release pharmaceutical preparations (for example, see International Publication Pamphlet WO 01/78686 or International Publication Pamphlet WO 93/05771), microparticle pharmaceutical preparations [for example, see Japanese Translation Publication (Kohyo) No. 10-511957], or mucous membrane adhesion-type pharmaceutical preparations [for example, see Japanese Unexamined Patent Publication (Kokai) No. 5-132416]. Preferred preparations are a hydrogel-forming sustained-release pharmaceutical preparation disclosed in International Publication Pamphlet WO 94/06414, a pharmaceutical preparation prepared by coating tablets of the above mixture with a high molecular substance that is dissolved in an organic acid, if desired, followed by a nonionic substance such as hydroxypropyl methyl cellulose, and further an enteric substance, or a timed-release pharmaceutical preparation disclosed in International Publication Pamphlet WO 95/28963.

### EXAMPLES

The present invention will now be further illustrated by, but is by no means limited to, the following Examples.

### Example 1

### 1. Substance to be Tested

Cefdinir obtained from Astellas Toyama was used as a test substance.

After 1000 g of aminoalkyl methacrylate copolymer E (Eudragit™ E100; Röhm GmbH) and 100 g of polysorbate 80 (Tween 80) were dissolved in 6000 g of 95% ethanol, 2000 g of 1N hydrochloric acid was added thereto. The whole was mixed, and spray-dried (inlet temperature: 85° C., outlet temperature: 65° C., spraying rate: 30 g/min.) using a spray-dryer (L-8 type; Ohkawara Kakohki Co., Ltd.) to obtain a spray-dried product (hereinafter referred to as E-SD).

As other reagents, those having a grade corresponding to guaranteed reagents or higher were used.

### 2. Formulation of Pharmaceutical Preparations

A 10 mmol/L phosphate buffer (pH 7.0) supplemented with 5% mannitol was prepared. E-SD was dissolved in this buffer at final concentrations of 0 μg/mL, 600 μg/mL, and 1800 μg/mL. To each E-SD solution, cefdinir was dissolved so as to become a concentration of 600 μg/mL.

### 3. Animals

SD rats (male, 7-week-old) were purchased from Japan SLC, Inc. The SD rats were bred for acclimation for about a week.

### 4. Dose, Administration, and Blood Collection

### 4.1 Dose

A dose was 10 mg/kg. Each cefdinir solution containing 600 μg/mL of cefdinir was administered at a dose of 16.7 mL/kg.

### 4.2 Preparations to be Administered

As a control preparation, a solution containing 600 μg/mL cefdinir (hereinafter referred to as “E-SD 0”) was used. As test preparations, a solution containing 600 μg/mL cefdinir and 600 μg/mL E-SD (hereinafter referred to as “E-SD 1”) and a solution containing 600 μg/mL cefdinir and 1800 μg/mL E-SD (hereinafter referred to as “E-SD 3”) were used.

### 4.3 Route and Method of Administration

Each rat that had been fasted for 16 hours or more was anesthetized with ether, and the abdomen was incised. A intestinal loop was formed by ligating at the Treitz's ligament and the ileocecal junction. A preparation to be tested was administered at a dose of 16.7 mL/kg, using a 5-mL syringe with a 26 G needle, from the ligation point immediately below the Treitz's ligament. After the administration, the intestinal loop was put back into the abdominal cavity, and the abdomen was closed by autoclips.

### 4.4 Blood Collection

Blood was collected from a jugular vein of each rat under ether anesthesia, using a heparinized syringe with a 26 G needle. Blood collections were carried out after 0.5 hour, 1 hour, and 2 hours from the administration, and the volume of blood to be collected was approximately 1 mL.

### 5. Storage of Samples

The collected bloods were centrifuged (10000 rpm, 4 min, 5° C.) to obtain plasma samples, which were kept at −20° C. or less until the following assay.

### 6. Assay of Samples

The extraction of cefdinir from the rat plasma samples and the determination thereof were carried out by the following procedures. In this connection, a standard curve (range of concentration: 50 ng/mL to 10 μg/mL, weight function: 1/X²) was made by preparing a diluted series of cefdinir, which were prepared by dissolving 5 mg of cefdinir in 100 mL of deionized water (MilliQ water) to obtain a stock solution (50 μg/mL, kept in a refrigerator) and diluting the stock solution with deionized water in a stepwise fashion.

Cefdinir was extracted from each rat plasma sample by a solid-phase extraction method. More particularly, rat plasma (0.2 mL), to which deionized water (0.1 mL) and 1 mol/L H₃PO₄ (0.4 mL) had been added, was applied to a cation-exchange column (Bond Elut SCX 100 mg; Varian). The column was washed with 0.01N HCl (2 mL) followed by deionized water (1 mL), and an elution was carried out using 0.2 mol/L Na₂HPO₄ (0.5 mL). An aliquot (100 μL) of the eluted solution was used for a quantitative analysis by HPLC. A standard curve was made by adding each aqueous solution containing a predetermined concentration of cefdinir (0.1 mL) and 1 mol/L H₃PO₄ (0.4 mL) to rat blank plasma (0.2 mL) to prepare standard solutions, and applying these standard solutions to the solid-phase extraction method and the quantitative analysis by HPLC as described above.

The HPLC was carried out under the following conditions.
Column: TSK gel ODS-80™ (Tosoh Corporation)

4.6 mm×25 cm
Mobile phase:

50 mmol/L phosphate buffer (pH 3):CH₃CN=85:15
Column temperature: 40° C.
Flow rate: 0.8 mL/min
UV: 280 nm

### 7. Results

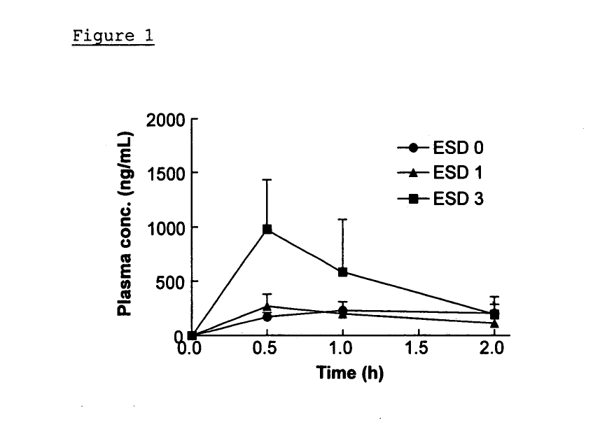

With respect to the results after the administration of each cefdinir solution with E-SD to the rat intestinal loop, time courses in mean plasma concentrations (mean±SD; n=3) are shown in FIG. 1, and pharmacokinetic parameters (mean±SD; n=3) are shown in Table 1. The data obtained in this experiment were processed by using a chromatographic data management system (Millennium 32; Waters) and a spreadsheet software (Microsoft Excel 2000-Ver.9.0; Microsoft).

TABLE 1

| | Tmax | Cmax | AUC_{0-2 h} | vs |
|---|---|---|---|---|
| | (h) | (ng/mL) | (ng · h/mL) | Control AUC_{0-2 h} |
| E-SD 0 | 1.33 ± 0.6 | 241 ± 67 | 362 ± 113 | 1.00 |
| E-SD 1 | 0.50 ± 0.0 | 270 ± 111 | 342 ± 80 | 0.94 |
| E-SD 3 | 0.50 ± 0.0 | 979 ± 452 | 1021 ± 662 | 2.82 |

With respect to plasma cefdinir concentrations after the administration of the E-SD containing solutions [i.e., the solution containing E-SD in the same amount as that of cefdinir by weight (E-SD 1), and the solution containing E-SD in an amount three times as much as that of cefdinir by weight (E-SD 3)], mean Cmax (maximum plasma cefdinir concentration) values were 270 ng/mL and 979 ng/mL, and mean AUC₀₋₂ₕ (area under the curve between 0-2 hours from the administration) values were 342 ng·h/mL and 1021 ng·h/mL, respectively. In comparison with the plasma cefdinir concentration (Cmax: 241 ng/mL, AUC₀₋₂ₕ: 362 ng·h/mL) after the administration of the control preparation without E-SD (E-SD 0), the concentration in the E-SD 1 was the same, but the concentration in the E-SD 3 was higher. In particular, the AUC₀₋₂ₕ value in the E-SD 3 was 2.82 times in comparison with the value in the E-SD 0. In all of the E-SD 3 cases (n=3), individual plasma cefdinir concentrations were higher than those in the E-SD 0.

When E-SD was added in an amount three times as much as the amount of cefdinir, the AUC₀₋₂ₕ value was approximately 2.8 times and the absorption of cefdinir from the intestines was significantly improved. From the result obtained wherein no effect was observed when E-SD was added in the same amount as that of cefdinir, and subsequent experimentation, it was considered that the absorption of cefdinir would be critically improved when E-SD was added in an amount two or more times as much as the amount of cefdinir.

These results suggest that E-SD can be used to improve the absorption of cefdinir and to enhance the bioavailability of cefdinir, that is, that even if a content of cefdinir in a pharmaceutical preparation is lowered in comparison with that in conventional preparations, the pharmacological activity thereof (and the concentration in blood) similar to those of conventional preparations will be maintained to reduce side effects on the digestive system.

### INDUSTRIAL APPLICABILITY

The present invention may be applied to enhance the bioavailability (particularly, to improve the oral absorption) of cefdinir preparations.

Please add a new paragraph at page 1 after the title and insert a new section heading as follows:

What is claimed is:

1. A pharmaceutical composition comprising cefdinir or a pharmaceutically acceptable salt thereof and aminoalkyl methacrylate copolymer E.

2. The pharmaceutical composition according to claim 1, further comprising an acidic substance.

3. The pharmaceutical composition according to claim 1, wherein the aminoalkyl methacrylate copolymer E is pulverized.

4. The pharmaceutical composition according to claim 2, comprising cefdinir or a pharmaceutically acceptable salt thereof, the aminoalkyl methacrylate copolymer E, and an acidic substance, wherein the three components are brought together and at least the aminoalkyl methacrylate copolymer E and the acidic substance are uniformly mixed.

5. The pharmaceutical composition according to claim 4, wherein cefdinir or a pharmaceutically acceptable salt thereof, the aminoalkyl methacrylate copolymer E, and an acidic substance are uniformly mixed.

6. The pharmaceutical composition according to claim 1, wherein an amount of the aminoalkyl methacrylate copolymer E is two or more parts by weight with respect to one part by weight of cefdinir or a pharmaceutically acceptable salt thereof.

7. The pharmaceutical composition according to claim 2, wherein the acidic substance has a feature such that when 1 g of the acidic substance is dissolved in 50 mL of water, a pH of the solution is 6 or lower.

8. The pharmaceutical composition according to claim 2, wherein the acidic substance is contained in an amount which neutralizes 10% or more of basic groups contained in the aminoalkyl methacrylate copolymer E.

9. The pharmaceutical composition according to claim 2, comprising 2 to 500 parts by weight of the aminoalkyl methacrylate copolymer E with respect to one part by weight of cefdinir or a pharmaceutically acceptable salt thereof in an amount effective for treating or preventing disease, and the acidic substance in an amount which neutralizes 10% or more of basic groups contained in the aminoalkyl methacrylate copolymer E.

10. The pharmaceutical composition according to claim 2, comprising 2 to 500 parts by weight of the aminoalkyl methacrylate copolymer E with respect to one part by weight of cefdinir or a pharmaceutically acceptable salt thereof in an amount effective for treating or preventing disease, and 0.005 to 50 parts by weight of the acidic substance with respect to one part by weight of the aminoalkyl methacrylate copolymer E.

11. The pharmaceutical composition according to claim 2, wherein the aminoalkyl methacrylate copolymer E and the acidic substance are obtained by spray-drying or freeze-drying a solution and/or suspension thereof in a pharmaceutically acceptable solvent.

12. The pharmaceutical composition according to claim 2, wherein the aminoalkyl methacrylate copolymer E and the acidic substance are contained in a form of a solution and/or suspension thereof in a pharmaceutically acceptable solvent.

13. The pharmaceutical composition according to claim 1, wherein the form of the pharmaceutical composition is one, or two or more preparations selected from the group consisting of granules, tablets, capsules, a suspension, and a liquid formulation.

14. A method of enhancing a bioavailability of cefdinir or a pharmaceutically acceptable salt thereof comprising: mixing cefdinir or a pharmaceutically acceptable salt thereof with the aminoalkyl methacrylate copolymer E.

15. The method according to claim 14, wherein an acidic substance is further added in the mixing step.

16. The method according to claim 14, wherein the aminoalkyl methacrylate copolymer E is pulverized.

17. The method according to claim 14, wherein the enhancement of the bioavailability is an improvement of an oral absorption.
